(19) United States
(12) Patent Application Publication
(10) Pub. No.: US 2014/0227371 A1
(43) Pub. Date: Aug. 14, 2014

(54) EDIBLE COMPOSITION

(71) Applicants: Alan David Heath, Sharnbrook (GB); Dawn Jennae Mazzatti, Trumbull, CT (US); Charlotte Mary Walden, Sharnbrook (GB)

(72) Inventors: Alan David Heath, Sharnbrook (GB); Dawn Jennae Mazzatti, Trumbull, CT (US); Charlotte Mary Walden, Sharnbrook (GB)

(21) Appl. No.: 14/131,010

(22) PCT Filed: May 31, 2012
(86) PCT No.: PCT/EP2012/060290

§ 371 Date: Apr. 7, 2014

(30) Foreign Application Priority Data
Jul. 13, 2011 (EP) 11173882.9

(51) Int. Cl. A61K 33/30; A23L 2/52; A23L 1/304; A61K 31/385; A61K 31/445

(52) U.S. Cl. CPC A61K 33/30; A61K 31/385; A61K 31/445; A23L 1/304; A23L 2/52
USPC 424/643

(57) ABSTRACT

The present invention relates to edible compositions for the maintenance of normal liver function comprising zinc, 1-deoxynojirimycinand α-lipoic acid.

### TECHNICAL FIELD OF THE INVENTION

The present invention relates to an edible composition that provides health benefits. More particularly, the invention relates to an edible composition comprising a combination of naturally occurring materials which interact synergistically to provide health benefits associated with the maintenance of liver function.

### BACKGROUND TO THE INVENTION

In order to survive during periods of famine, mammals have evolved mechanisms to store energy during periods of plenty. When consumption of energy far exceeds the combustion of calories, the excess energy is stored as fat. Prolonged periods of positive energy balance leads to weight gain and obesity. Sedentary lifestyle and poor dietary choices have caused obesity to become epidemic in industrialised nations, and the prevalence of obesity in developing countries is also increasing rapidly. Obesity has been linked with increased likelihood of several diseases, e.g. insulin resistance, heart disease, hypertension, type 2 diabetes, liver disease and certain types of cancer.

Obesity has now overtaken alcohol as the number one cause of liver disease. Non-alcoholic fatty liver disease (NAFLD) refers to a spectrum of metabolic liver disorders which occur in people who do not drink alcohol excessively. At one end of this spectrum is simple fatty liver (accumulation of fat in the liver, also known as hepatic steatosis). Non-alcoholic steatohepatitis (NASH) is a significant development in NAFLD, with the fat in the liver now causing liver inflammation. This is a more aggressive condition that may cause scarring to the liver (fibrosis) and can ultimately progress to cirrhosis, causing irreversible liver damage.

Current management of NAFLD is largely conservative and focuses on reducing metabolic risk factors, with the mainstay of therapy focusing on lifestyle modifications such as gradual weight loss through diet and regular exercise. However, compliance in diet and lifestyle interventions is poor, with drop out rates typically around 30% to 40%. Therefore it would be desirable to identify alternative ways to prevent and/or ameliorate NAFLD.

Numerous dietary ingredients have been postulated to have beneficial effects with respect to hepatic steatosis. Several such ingredients and combinations thereof were investigated by the inventors, but the majority were not found to have a significant effect on the accumulation of lipids in hepatocytes.

Zinc has been studied extensively in experimental models of alcohol-induced steatosis and steatohepatitis, and has been reported to have hepatoprotective effects (for example, Kang et al. (2005) Mol Aspects Med 26: 391-404).

Several studies have indicated that α-lipoic acid administration might be beneficial in liver disease, especially alcoholic liver disease (for example, Marshall et al. (1982) Gut 23: 1088-1093).

1-Deoxynojirimycin (DNJ) has been reported to suppress lipid accumulation through activation of the β-oxidation system in rat liver (Tsuduki et al. (2009) J Agric Food Chem 57: 11024-11029).

The present inventors have surprisingly found that a combination of zinc, α-lipoic acid and DNJ can act synergistically to prevent lipid accumulation in liver cells.

### SUMMARY OF THE INVENTION

Thus in a first aspect, the present invention provides an edible composition comprising zinc, 1-deoxynojirimycin and α-lipoic acid.

Zinc is an essential trace element that is involved in numerous aspects of cellular metabolism. The composition comprises zinc, preferably in an amount of 0.1 to 40 mg, more preferably 0.5 to 30 mg, and most preferably 1 to 20 mg.

1-Deoxynojirimycin (DNJ), a constituent of mulberry leaves (Morus alba and/or Morus Bombysis), is a D-glucose analogue in which the oxygen atom of the pyranose ring is substituted by an NH group. The composition comprises 1-deoxynojirimycin, preferably in an amount of 0.5 to 1000 mg, more preferably 1 to 800 mg and most preferably 5 to 500 mg.

α-Lipoic acid (6,8-dithiooctanoic acid) is an antioxidant which functions as a co-factor for mitochondrial enzymes. The composition comprises α-lipoic acid, preferably in an amount of 5 to 2000 mg, more preferably 15 to 1500 mg and most preferably 40 to 800 mg.

A composition comprising zinc, 1-deoxynojirimycin and α-lipoic acid is believed to be of use to provide a number of health benefits. Thus in a second aspect, the present invention provides the composition of the first aspect of the invention for use as a medicament.

In particular, the composition may provide health benefits associated with the maintenance of liver function. It is thought that the accumulation of fat in the liver may lead to impaired liver function. Thus, in a third aspect, the present invention provides a composition comprising zinc, 1-deoxynojirimycin and α-lipoic acid for use in the treatment and/or prevention of hepatic steatosis. Furthermore, in a fourth aspect, the present invention provides a composition comprising zinc, 1-deoxynojirimycin and α-lipoic acid for use in the treatment and/or prevention of steatohepatitis.

Most individuals with hepatic steatosis will also have insulin resistance and/or type 2 diabetes. Therefore, in a fifth aspect, the present invention provides a composition comprising zinc, 1-deoxynojirimycin and α-lipoic acid for use in the treatment and/or prevention of insulin resistance. Furthermore, in a sixth aspect, the present invention provides a composition comprising zinc, 1-deoxynojirimycin and α-lipoic acid for use in the treatment and/or prevention of type 2 diabetes.

### FIGURES

By way of example, the invention is illustrated with reference to the following figure:

FIG. 1 shows relative lipid accumulation as a percentage of the control (untreated cells) for HepG2 cells treated for 48 hours with PA (treatment 1), PA+Zn (treatment 2), PA+LA (treatment 3), PA+DNJ (treatment 4), PA+Zn+LA (treatment 5), PA+Zn+DNJ (treatment 6), PA+LA+DNJ (treatment 7) and PA+Zn+LA+DNJ (treatment 8), wherein PA is 100 μM palmitic acid, Zn is 37.5 μM zinc, LA is 150 μM α-lipoic acid and DNJ is 75 μM 1-deoxynojirimycin.

### DETAILED DESCRIPTION

As used herein the term “comprising” encompasses the terms “consisting essentially of” and “consisting of”. All percentages and ratios contained herein are by weight of the final composition unless otherwise indicated. It should be noted that in specifying any range of values or amounts, any particular upper value or amount can be associated with any particular lower value or amount.

The Composition

The composition of the present invention is an edible composition, and as such is suitable for direct human consumption. The edible composition of the invention may be in the form of capsules, pills, tablets, granules, solutions, suspensions or emulsions. Preferably the edible composition is a food or a beverage product. Preparation of compositions of the invention in the aforementioned oral formats is well known to the person skilled in the art.

Food products of the invention are preferably part of the normal daily diet, for example margarines or other spreads or oil-based products, sauces especially dressings or mayonnaise, nutrition bars, soups, soluble powder products including those for preparing a beverage, or confectionery products, especially frozen confectionery products such as water-ice or ice cream.

As used herein the term beverage refers to a substantially aqueous drinkable composition suitable for human consumption. Preferably the beverage comprises at least 85% water by weight, more preferably at least 90% and most preferably from 95 to 99.9%. The beverage product may be cold or hot and may be a drink prepared from a powder, concentrated extract or syrup, or a drink sold in a ready to drink form. Examples of beverage products include soft drinks, fruit juices, tea-based drinks and/or soy-based drinks. The serving size of the composition (i.e. the amount of the edible composition that is intended to be consumed as a single portion) will depend on the format of the composition. For beverages a typical serving size is 50 to 500 ml, preferably 100 to 400 ml, more preferably 200 to 350 ml.

In a preferred embodiment the edible composition is packaged (i.e. contained within a sealed package). Examples of suitable packages include bottles, cans, cartons, pouches and sachets. In particular, from a standpoint of microbial stability, it is preferred that the package is sealed such that it is impermeable to microbiological contaminants. For example, where the edible composition is a liquid composition (e.g. a beverage product) it is desirable that the packaged composition can be stored for at least 6 months at a temperature of 20° C. without the amount of spore-forming bacteria (Bacillus and Clostridia spp.) in the composition increasing above 100 cfu/ml.

The edible composition comprises zinc, 1-deoxynojirimycin and α-lipoic acid. Preferably, the amount of zinc, 1-deoxynojirimycin and α-lipoic acid in a single serving of the composition will not exceed the recommended daily limit for an adult person.

The naturally occurring isomer of α-lipoic acid (LA) is R-α-lipoic acid (R-LA). However, virtually all of the published studies of LA supplementation in humans have used racemic LA. After oral dosing with racemic LA, peak plasma concentrations of R-LA are found to be higher than S-LA, suggesting R-LA is better absorbed than S-LA (Hermann et al. (1996) Eur J Pharm Sci 4: 167-174). However, both isomers of LA are metabolised and excreted (Teichert et al. (2003) J Clin Pharmacol 43: 1257-1267). Therefore, the present composition may comprise racemic α-lipoic acid and/or R-α-lipoic acid.

The molar ratio of zinc to 1-deoxynojirimycin is preferably 10:1 to 1:50, more preferably 5:1 to 1:20 and most preferably 2:1 to 1:10. The molar ratio of zinc to α-lipoic acid is preferably 10:1 to 1:50, more preferably 5:1 to 1:20 and most preferably 2:1 to 1:10. The molar ratio of 1-deoxynojirimycin to α-lipoic acid is preferably 20:1 to 1:20, more preferably 10:1 to 1:10 and most preferably 5:1 to 1:5.

Health Benefits

The liver is a central player in maintaining whole body energy homeostasis, largely due to its role in regulating the levels of glucose and free fatty acids in the bloodstream. When energy intake is abundant, mammals preferentially burn dietary carbohydrates to generate adenosine triphosphate (ATP) and surplus glucose. The liver stores some of this glucose as glycogen (via glycogenesis). When the liver is saturated with glycogen (roughly 5% of liver mass), additional glucose is shunted into pathways leading to lipid synthesis. The liver is also able to convert stored fuels into glucose in order to keep blood glucose levels stable during periods of starvation. The production of glucose by the liver is finely tuned by hormone signals and involves two simultaneous and ongoing pathways: glycogenolysis (breakdown of glycogen stores to produce glucose) and gluconeogenesis (de novo synthesis of glucose from pyruvate).

Insulin is usually considered to be the dominant regulator of glucose output from the liver. In the post-absorptive state, circulating levels of insulin drop and hepatic glucose production pathways (i.e. glycogenolysis and gluconeogenesis) are activated to in order to regulate fasting plasma glucose concentrations and provide a sufficient supply of glucose to the central nervous system. In the postprandial period, insulin levels rise, stimulating both glycogenesis (synthesis of glycogen) and glycolysis (conversion of glucose into pyruvate), while simultaneously suppressing hepatic glucose production pathways. This results in a net hepatic uptake of glucose, which in turn restricts postprandial increases in plasma glucose concentrations. In addition, when glucose concentrations are elevated, the liver has the ability to synthesise lipids de novo from glucose via glycolysis and lipogenesis.

Thus the maintenance of liver function is paramount to human health, particularly with respect to the prevention of metabolic diseases (e.g. insulin resistance, type 2 diabetes). It is thought that the accumulation of fat in the liver may lead to impaired liver function. Therefore, a composition which can prevent the accumulation of fat in the liver is likely to be useful in providing health benefits associated with the maintenance of liver function. Therefore the present invention provides a composition comprising zinc, 1-deoxynojirimycin and α-lipoic acid for use as a medicament.

Liver Function

Liver function can be monitored via blood tests which measure the level of certain serological markers. These serological markers are commonly referred to as “liver enzymes” and the blood tests used to measure them as “liver enzyme tests”. For example, a commonly used test involves the determination of serum alanine aminotransferase (ALT) and aspartate aminotransferase (AST) levels. These enzymes are present in hepatocytes (liver cells) and leak out when these cells are damaged. Normal levels of liver enzymes indicate that an individual has normal liver function, whereas elevated levels of liver enzymes indicate that an individual has impaired liver function.

Fatty Liver Disease

The primary event of hepatic steatosis (fatty liver disease) is the accumulation of triglycerides (i.e. fat) in hepatocytes. In certain individuals, hepatic steatosis can progress to steatohepatitis. The present inventors have surprisingly found that a combination of zinc, 1-deoxynojirimycin and α-lipoic acid can act synergistically to prevent the accumulation of triglycerides in hepatocytes.

Thus, it is envisaged that a composition according to the present invention can be used in the manufacture of a medicament for the treatment or prevention of hepatic steatosis and/or steatohepatitis. Furthermore, the present invention also relates to a method for the treatment or prevention of hepatic steatosis and/or steatohepatitis in a person in need thereof comprising the step of administering an effective amount of a composition according to the present invention.

Insulin Resistance

Insulin resistance refers to a reduced responsiveness of a target cell or a whole organism to the insulin concentration to which it is exposed. Insulin resistance is thought to be the key primary defect underlying the development of type 2 diabetes.

Insulin resistance is an almost universal finding in fatty liver disease, and manifests as a reduction in the efficiency of insulin to inhibit hepatic glucose production. Insulin resistance plays an important role in the development and progression of fatty liver disease, and the severity of insulin resistance appears to predict the likelihood of hepatic steatosis progressing to steatohepatitis. Despite the existing correlation between fatty liver and insulin resistance, the mechanisms linking hepatic fat and insulin resistance have not been fully elucidated. It remains unclear whether insulin resistance causes accumulation of fat in the liver, or whether the increase in hepatic lipid content may itself play a causal role in the development of insulin resistance. Nevertheless, a composition which can protect the liver against lipid accumulation will likely be beneficial in the treatment or prevention of insulin resistance.

Thus it is envisaged that a composition according to the present invention can be used in the manufacture of a medicament for the treatment or prevention of insulin resistance. Furthermore, the present invention also relates to a method for the treatment or prevention of insulin resistance in a person in need thereof comprising the step of administering an effective amount of a composition according to the present invention.

Type 2 Diabetes

Type 2 diabetes (formerly called non-insulin-dependent or adult-onset diabetes) results from the body's ineffective use of insulin. The current World Health Organisation diagnostic criteria for diabetes are: a fasting plasma glucose ≧7.0 mmol/l (126 mg/dl) or a 2 h plasma glucose 11.1 mmol/l (200 mg/dl).

In type 2 diabetes, alterations in hepatic glucose metabolism are observed. Post-absorptive glucose production is increased and postprandial suppression of glucose production is impaired. In addition, the simultaneous overproduction of glucose and lipids in the liver further stimulates the secretion of insulin by pancreatic β-cells. Elevated insulin levels promote glycolysis and lipogenesis in the liver, thereby establishing a vicious circle. Without wishing to be bound by theory, we believe that the composition of the present invention has the potential to reduce the production of lipids in the liver and thereby break this cycle.

Thus it is envisaged that a composition according to the present invention can be used in the manufacture of a medicament for the treatment or prevention of type 2 diabetes. Furthermore, the present invention also relates to a method for the treatment or prevention of type 2 diabetes in a person in need thereof comprising the step of administering an effective amount of a composition according to the present invention.

The present invention will now be illustrated by reference to the following non-limiting example.

### EXAMPLE 1

Materials

The materials used to generate the data described herein are listed in table 1.

TABLE 1

| Material | Supplier | Catalogue No. | Notes |
|---|---|---|---|
| HepG2 cells | ATCC | HB-8065 | Hepatocellular carcinoma, |
| | | | human |
| Eagle's Minimum | Lonza | BE12-125F | Contains Earle's balanced salt |
| Essential Medium | | | solution and non-essential |
| (EMEM) | | | amino acids |
| Fetal bovine serum (FBS) | Invitrogen | 10500-056 | Heat inactivated |
| L-Glutamine solution | Sigma-Aldrich | G7513 | 200 mM, sterile filtered |
| Dulbecco's PBS | Sigma-Aldrich | D8537 | |
| Trypsin-EDTA solution | Sigma-Aldrich | T3924 | 1x, sterile filtered |
| Palmitic acid | Sigma-Aldrich | P0500 | Stock solution (20 mM) prepared |
| | | | in 100% (v/v) methano |
| α-Lipoic acid | Sigma-Aldrich | T1395 | Stock solution (30 mM) prepared |
| | | | in 80% (v/v) methano |
| 1-Deoxynojirimycin | Sigma-Aldrich | D9305 | Stock solution (15 mM) prepared |
| hydrochloride | | | in 80% (v/v) methanol |
| Zinc sulphate | Sigma-Aldrich | Z0251 | Stock solution (50 mM) prepared |
| | | | in distilled water |
| Cell Proliferation | Roche | 11644807001 | |
| Reagent WST-1 | | | |
| Steatosis Colorimetric | Cayman Chemical | 10012643 | |
| Assay Kit | | | |

Cell Culture

HepG2 cells were routinely cultured using aseptic technique. Cells were grown in a humidified incubator at 37° C., 5% (v/v) CO₂. Culture medium was EMEM supplemented with 10% (v/v) FBS and 2 mM L-glutamine and was replaced 2 to 3 times per week. Confluent cells were subcultured by trypsinisation according to the protocol recommended by ATCC.

Treatment of Cells

HepG2 cells were plated in 96 well plates at a seeding density of approximately 12,000 cells per well. The cells were grown in low serum medium (i.e. EMEM supplemented with 0.5% (v/v) FBS and 2 mM L-glutamine) for 24 hours prior to treatment.

In order to induce steatosis, the HepG2 cells were treated with 100 μM palmitic acid (PA) for 48 hours. A control sample was included wherein steatosis was not induced (i.e. the cells were cultured in the absence of palmitic acid). Where appropriate, zinc (Zn), α-lipoic acid (LA) and/or 1-deoxynojirimycin (DNJ) were added to the culture medium at the same time as the palmitic acid (see table 2). The concentration of vehicle (i.e. methanol) was kept constant across the treatments and was never more than 1% (v/v) in any treatment. In order to minimise edge effects the outer wells of the plates were not used for treatments and different well positions were used for the treatments in repeat experiments.

TABLE 2

| | Palmitic | | α-Lipoic | 1-deoxynojirimycin |
|---|---|---|---|---|
| Treatment | acid (PA) | Zinc (Zn) | acid (LA) | (DNJ) |
| Control | — | — | — | — |
| 1 | 100 μM | — | — | — |
| 2 | 100 μM | 37.5 μM | — | — |
| 3 | 100 μM | — | 150 μM | — |
| 4 | 100 μM | — | — | 75 μM |
| 5 | 100 μM | 37.5 μM | 150 μM | — |
| 6 | 100 μM | 37.5 μM | — | 75 μM |
| 7 | 100 μM | — | 150 μM | 75 μM |
| 8 | 100 μM | 37.5 μM | 150 μM | 75 μM |

Cell Viability Assay

Following the 48 hour treatment, cell viability was determined using a colorimetric assay. The assay was performed using the WST-1 Cell Proliferation Reagent (Roche) as per the manufacturer's instructions (Version 14: October 2007). Briefly, the culture medium was removed and the cells were washed once with PBS. The WST-1 reagent (diluted 1:10 in culture medium) was added to the cells, which were then incubated for 50 minutes at 37° C., 5% (v/v) CO₂. Reduction of the WST-1 reagent by viable cells during this incubation period produces a soluble formazan salt, which can be quantified by measuring absorbance at 450 nm against a background control (i.e. diluted WST-1 reagent in the absence of cells) as blank. The measured absorbance directly correlates with the number of viable cells.

Steatosis Assay

Following the cell viability assay, the cell viability reagent was removed and the cells were washed once with PBS. The accumulation of lipid within the HepG2 cells was determined by using Oil Red O to stain neutral lipids. The staining procedure was performed using the Steatosis Colorimentric Assay Kit (Cayman Chemical) as per the manufacturer's instructions. Lipid accumulation was quantified by extracting the dye from the cells using the Dye Extraction Solution and the absorbance was read at 450 nm.

Results

The lipid accumulation was normalised to cell viability by dividing the lipid accumulation value by the cell viability value. The mean lipid accumulation of the control sample was defined as being 100%. The relative lipid accumulation (as a percentage of the control sample) for each of the treatments was then calculated.

TABLE 3

| | | | | | Relative lipid | Standard |
|---|---|---|---|---|---|---|
| Treatment | PA | Zn | LA | DNJ | accumulation (% control) | deviation |
| Control | — | — | — | — | 100 | 22 |
| 1 | Y | — | — | — | 197 | 58 |
| 2 | Y | Y | — | — | 169 | 75 |
| 3 | Y | — | Y | — | 154 | 64 |
| 4 | Y | — | — | Y | 209 | 73 |
| 5 | Y | Y | Y | — | 165 | 77 |
| 6 | Y | Y | — | Y | 294 | 172 |
| 7 | Y | — | Y | Y | 125 | 37 |
| 8 | Y | Y | Y | Y | 92 | 39 |

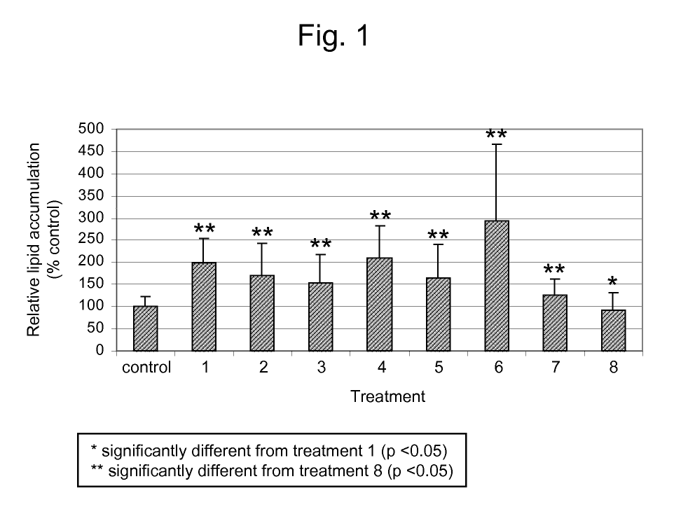

The results obtained for the control and for treatments 1 to 8 are summarised in table 3 (n=6 in all instances), and are also represented graphically in FIG. 1. Treatments 1 to 7 are comparative examples (i.e. these treatments concern compositions outside the scope of the present invention), while treatment 8 relates to a composition according to the present invention.

The data were statistically analysed using a one-way ANOVA (Kruskal-Wallis method) and a pair-wise multiple comparison procedure (Student-Newman-Keuls method) (table 4). The results show that 100 μM palmitic acid induced lipid accumulation in hepatocytes (treatment 1). Moreover, treatment of hepatocytes with any one of zinc, α-lipoic acid or 1-deoxynojirimycin in addition to palmitic acid (i.e. treatments 2, 3 and 4, respectively) did not have a significant effect on lipid accumulation compared to treatment 1. Similarly, treating hepatocytes with combinations of any two of these ingredients (i.e. treatments 5, 6 and 7) in addition to palmitic acid did not significantly change lipid accumulation compared to treatment 1. Indeed, table 4 shows that the only treatment in which the relative lipid accumulation in hepatocytes was significantly reduced (p<0.05) compared to treatment 1 was treatment 8 (i.e. treatment of cells with a combination of zinc, α-lipoic acid and 1-deoxynojirimycin in addition to palmitic acid). Furthermore, the relative lipid accumulation in hepatocytes was significantly increased (p<0.05) for any of treatments 1 to 7 when compared to treatment 8.

TABLE 4

| | | | | | Sig diff from | Sig diff from |
|---|---|---|---|---|---|---|
| Treatment | PA | Zn | LA | DNJ | treatment 1 | treatment 8 |
| 1 | Y | — | — | — | | p < 0.05 |
| 2 | Y | Y | — | — | | p < 0.05 |
| 3 | Y | — | Y | — | | p < 0.05 |
| 4 | Y | — | — | Y | | p < 0.05 |
| 5 | Y | Y | Y | — | | p < 0.05 |
| 6 | Y | Y | — | Y | | p < 0.05 |
| 7 | Y | — | Y | Y | | p < 0.05 |
| 8 | Y | Y | Y | Y | p < 0.05 | |

1. An edible composition comprising 0.1 to 40 mg zinc, 0.5 to 1000 mg 1-deoxynojirimycin and 5 to 2000 mg α-lipoic acid.

2. A composition as claimed in claim 1 wherein the composition comprises 0.5 to 30 mg of zinc, preferably 1 to 20 mg.

3. A composition as claimed in claim 1- wherein the composition comprises 1 to 800 mg of 1-deoxynojirimycin, preferably 5 to 500 mg.

4. A composition as claimed in claim 1 wherein the composition comprises 15 to 1500 mg of α-lipoic acid, preferably 40 to 800 mg.

5. A composition as claimed in claim 1 wherein the molar ratio of zinc to 1-deoxynojirimycin is 10:1 to 1:50, preferably 5:1 to 1:20, more preferably 2:1 to 1:10.

6. A composition as claimed in claim 1 wherein the molar ratio of zinc to α-lipoic acid is 10:1 to 1:50, preferably 5:1 to 1:20, more preferably 2:1 to 1:10.

7. A composition as claimed in claim 1 wherein the molar ratio of 1-deoxynojirimycin to α-lipoic acid is 20:1 to 1:20, preferably 10:1 to 1:10, more preferably 5:1 to 1:5.

8. A composition as claimed in claim 1, wherein the composition is a food or beverage product.

9. A composition as claimed in claim 1 wherein the composition is packaged.

10. A composition comprising 0.1 to 40 mg zinc, 0.5 to 1000 mg 1-deoxynojirimycin and 5 to 2000 mg α-lipoic acid for use as a medicament.

11. A composition comprising 0.1 to 40 mg zinc, 0.5 to 1000 mg 1-deoxynojirimycin and 5 to 2000 mg α-lipoic acid for use in the treatment and/or prevention of hepatic steatosis.

12. A composition comprising 0.1 to 40 mg zinc, 0.5 to 1000 mg 1-deoxynojirimycin and 5 to 2000 mg α-lipoic acid for use in the treatment and/or prevention of steatohepatitis.

13. A composition according to claim 10 wherein the composition comprises 0.5 to 30 mg of zinc, preferably 1 to 20 mg.

14. A composition according to claim 10 wherein the composition comprises 1 to 800 mg of 1-deoxynojirimycin, preferably 5 to 500 mg.

15. A composition according to claim 10 wherein the composition comprises 15 to 1500 mg of α-lipoic acid, preferably 40 to 800 mg.
